(19) United States
(12) Patent Application Publication
(10) Pub. No.: US 2017/0216059 A1
(43) Pub. Date: Aug. 3, 2017

(54) METHODS AND APPARATUS FOR INCREASING THE PROXIMAL MOVEMENT OF BLOOD OR LYMPH

(71) Applicants: James E. SCHROEDER, San Antonio, TX (US); Nicolas E. WALSH, San Antono, TX (US)

(72) Inventors: James E. SCHROEDER, San Antonio, TX (US); Nicolas E. WALSH, San Antono, TX (US)

(73) Assignee: THE BOARD OF REGENTS OF THE UNIVERSITY OF TEXAS SYSTEM, Austin, TX (US)

(21) Appl. No.: 15/302,064

(22) PCT Filed: Apr. 17, 2015
(86) PCT No.: PCT/US15/26550

§ 371 Date: Oct. 5, 2016

Related U.S. Application Data
(60) Provisional application No. 61/980,837, filed on Apr. 17, 2014.

(51) Int. Cl. A61F 2/80; A61F 5/01; A61F 2/78

(52) U.S. Cl. CPC A61F 2/80; A61F 2/7812; A61F 5/01; A61F 2002/7818; A61F 2002/785

(57) ABSTRACT

Embodiments are directed to devices configured to assist the flow of fluids in the body. The device comprises a covering for placing on the skin of a patient having protrusions which can contact the skin. The protrusions have a base proximal to a surface of the covering and an outer edge distal to the surface of the covering. Protrusions can be configured in an array, in a line perpendicular to desired fluid flow or in a spiral configuration, such that fluid flow is enhanced in the appropriate direction. The device can be configured as a prosthesis, an orthotic, a liner for a prosthesis or orthotic, or a wrap or covering that is positioned around a body part. The body part can be part of a lower limb, part of an upper limb, or other body part.

This Application claims priority to U.S. Provisional Patent Application Ser. No. 61/980,837 filed Apr. 17, 2014, which is incorporated herein by reference in its entirety.

### BACKGROUND

Various conditions can result in the pooling of blood and/or lymph in tissues including injury, disease, amputation, and the surgical removal of lymph nodes. Persons with fluid accumulation may suffer from fatigue, pain, discomfort, discoloration, elephantiasis, and an increased risk of infection. Currently there are no effective means to transport the fluid out of the affected tissue.

Individuals born with lower limb loss or those who have had an amputation through injury, infection, or disease are traditionally fit with a prosthetic socket and limb so as to provide them with the opportunity to achieve independent ambulation. In order for a patient to successfully stand and ambulate the prosthetic socket must transfer the loads that are normally distributed through the skeletal system to the residual limb and its delicate soft tissues. A prosthetic socket typically has an intimate fit with the residual limb in order to effectively and safely transfer these forces.

Fluid management within a residual limb becomes a problem during the wearing of a prosthetic device by an amputee. Loss of fluid from the residual limb can occur during the course of a day or over a longer period of time, causing fit problems with the prosthetic device. In addition, prosthetic devices may also cause swelling and edema problems in residual limbs due to pooling of fluid. The effects of pressure, both positive and negative, within the prosthesis may compound these potential problems. Amputees also can encounter vascular flow problems in the residual limb. Sometimes vascular problems are the reason for the amputation in the first place, for example, when patients with diabetes develop complications in a lower limb partially due to poor circulation. Such vascular problems may slow the proper healing of sustained wounds (e.g., irritations, rashes, cuts, punctures, bruises, lacerations, abrasions, injuries, etc.), or of ulcers that have developed on the residual limb. While assuring that a residual limb has an adequate blood supply is always important, the results of a recent study suggested that in some cases, it is the timely evacuation of deoxygenated, nutrient-depleted, and waste-rich “spent blood” from the limb that also is important for residual limb health. Timely removal of spent blood can improve healing and the overall health of the limb by improving overall circulation and indirectly increasing the supply of oxygenated, nutrient-rich, and waste-removed blood to the tissues in the limb.

For amputees, management of fluid within a residual limb is important for fit and comfort reasons. In addition, management of fluid in the body tissue is also important for proper vascular and lymphatic flow in other diseases and can promote wound healing in other diseases and conditions having an associated vascular or lymphatic dysfunction, e.g., diabetes, peripheral artery disease, edema, lymphedema, etc.

The lymphatic system consists of lymph vessels, lymph nodes and lymphoid tissues and is a secondary system within the circulatory system that removes waste. Unlike the closed-loop blood circulatory system, the lymphatic system works according to a one-way principal. That is, the lymphatic system is a drainage system to drain away lymph that continually escapes from the blood in small amounts. The lymph is first collected at the lymph capillaries, which in turn drain into larger vessels. The lymph is pumped in and out of these vessels by movements of adjacent muscles and by contractions of the walls of the larger vessels, and moves through the lymphatic system in one direction. Foreign matter and bacteria are filtered at various lymph node groups after which the fluid empties into the venous portion of the blood system, mainly through the thoracic duct. A healthy person will drain one to two liters of lymph fluid through this duct every day. Without proper drainage into the duct, lymphedema results.

Lymphedema is an accumulation of a watery fluid in the body causing a swelling or edema of the affected area. The swelling causes pain, discomfort, disfigurement and interference with wound healing and, if left untreated, can cause fibrosis. Fibrosis is a hardening of the tissue in the affected area, which may further complicate the drainage process and can cause life-threatening conditions, such as infections. Lymphedema may be congenital or may result from surgery when the lymph nodes are removed in order to prevent the further spread of cancerous conditions, such as with a mastectomy or prostatectomy, and may also be caused by filariasis.

In a further aspect pooling of blood and/or lymph in tissues can be a result of illness, accident, or immobilization resulting from bodily injury. Bed-ridden or wheelchair-bound patients who are unable to perform even a modest amount of movement frequently develop ulcers and other complications that can result from the development of necrotic tissue. Patients with immobile limbs, such as elderly, debilitated or sedated patients as well as diabetics can suffer significant skin damage due to circulatory deficiency and are particularly prone to pressure ulcers after only a few hours of immobile bed rest. A continued lack of adequate blood flow, and the resulting lack of oxygen, can lead to cell death and necrosis.

There remains a need for additional methods and devices to assist fluid movement and prevent pooling of fluids.

### SUMMARY

Increased movement of fluids such as blood or lymph toward the heart would benefit the health of patients having vascular dysfunction (e.g., diabetics), patients for whom the normal mechanisms for returning blood are compromised (e.g., amputees), and patients who have edema or lymphedema. Other patients who can benefit from the devices described are hemiplegics with one hemiparetic limb and patients with chronic venous insufficiency. Fluid movement also helps with the healing process for extremities in general by the timely removal of waste materials (lymph) and spent blood, and by increasing overall blood circulation to the affected region.

Certain embodiments are directed to prostheses configured to assist the movement of fluids. In certain aspects a prosthesis comprises a prosthetic socket configured to receive a residual limb, including any coverings, sleeves, and/or socks coverings. A covering will allow intermittent contact of the protrusions or patterned surface with the skin. In certain aspects the wall of the socket has protrusions formed on the surface. The protrusions having a base proximal to the socket wall and an outer edge distal to the wall, i.e., protruding into the socket. In certain aspects the protrusions have a first acute angle relative to the inner skin-facing surface of the prosthetic socket or device positioned such that the apex of the angle is position in the direction of desired fluid flow. In certain aspects the first angle can be less than or about 60, 55, 50, 45, 40, 35, 30, 25, 20, 15 degrees, including all values and ranges there between, relative to the plane of the socket wall. In a further aspect the first angle is between 15 and 30 degrees relative to the plane of the socket wall. In still a further aspect the first angle is approximately 20 degrees relative to the plane of the socket wall. In certain embodiments the protrusion is a wedge or a flap. In certain aspects the protrusion will have a substantially perpendicular second angle positioned at the bottom edge side of the protrusion. In other aspects the protrusion can be configured to have a hinge mechanism to allow the protrusion to move toward and away from the socket wall. The edge of the protrusion is to be positioned perpendicular to fluid flow with the acute angle positioned on the side of protrusion directed to the desired direction of fluid flow. In certain aspects the protrusion edge is rounded. The protrusion is configured such that when the protrusion comes in contact with a body part or a covering of a body part a rolling force towards the heart is generated. Protrusions can be configured in an array, in a line perpendicular to desired fluid flow or in a spiral configuration, such that fluid flow is enhance in the appropriate direction. The protrusions on the wall of the prosthesis will, with activity, move toward and away from the limb covering imparting desired “milking” pressure changes either directly to the surface of the skin or indirectly (through any covering such as a sock or liner) to the surface of the skin as a user walks and performs activities of daily living (ADLs).

In certain embodiments the device is an orthotic device or orthosis. Orthotics is a medical specialty concerned with the design, manufacture, and application of externally applied devices used to modify the structural and functional characteristics of the neuromuscular and skeletal system, i.e. orthosis. An orthosis may be used to: (i) control, guide, limit and/or immobilize an extremity, joint or body segment for a particular reason; (ii) restrict movement in a given direction; (iii) assist movement generally; (iv) reduce weight bearing forces for a particular purpose; (v) aid rehabilitation from fractures after the removal of a cast; (vi) otherwise correct the shape and/or function of the body providing easier movement capability or reduce pain. Patients benefiting from an orthosis may have a condition such as spina bifida or cerebral palsy, or have experienced a spinal cord injury or stroke. Equally, orthoses are sometimes used prophylactically or to optimise performance in sport. In certain asepcts the orthosis or orthotic is a configured as a covering or sleeve that is pulled over or positioned around a body part. The body part can be part of a lower limb, such as a foot, calf, thigh, or leg; or an upper limb such as a hand, wrist, forearm, upper arm, or arm. In a further aspect body part can be all or part of the neck, chest, torso, or waist. In certain aspects the orthotic is configured as a wrap having a first and second side edges that are configured to be attached to each other to form the lumen. The protrusions can be configured as linear bands, spiral bands, or an array of protrusions; or the size of the protrusions are heterogeneous. In certain aspects spatial distribution of the protrusions associated with an orthosis is irregular.

Other embodiments are directed to vascular assist devices comprising a pliable sheet or lining, which can be in the form of a liner positioned within a prosthetic socket, having an interior surface and exterior surface, the interior surface comprising protrusions having a base proximal to the interior surface and an outer edge distal to the interior surface. The sheet or lining can be removably positioned in the socket of a prosthesis. In certain aspects the protrusions have a first acute angle relative to the inner surface positioned such that the apex of the angle is position in the direction of desired fluid flow. In certain aspects the first angle can be less than or about 60, 55, 50, 45, 40, 35, 30, 25, 20, 15 degrees, including all values and ranges there between, relative to the plane of the interior surface. In a further aspect the first angle is between 15 and 30 degrees relative to the plane of the interior surface. In still a further aspect the first angle is approximately 20 degrees relative to the plane of the interior surface. In certain embodiments the protrusion is a wedge or a flap. In certain aspects the protrusion will have a substantially perpendicular second angle positioned at the bottom edge side of the protrusion. In other aspects the protrusion can be attached to the interior surface and configured to have a hinge mechanism to allow the protrusion to move toward and from the centerline of the sheet or lining. The edge of the protrusion is to be positioned perpendicular to fluid flow with the acute angle positioned on the side of protrusion directed to the desired direction of fluid flow. In certain aspects the edge is rounded. When the protrusion comes in contact with a body part of a subject a rolling force towards the heart is generated. Protrusions can be configured in an array, in a line perpendicular to desired fluid flow or in a spiral configuration, such that fluid flow is enhance in the appropriate direction.

In still other embodiments the pliable sheet is configured as a covering or sleeve that is pulled or rolled over a body part. The covering or sleeve can have one or two open ends, i.e., one end can be a closed end. In certain aspects the body part is a residual limb, a normal limb, an injured limb, or a diseased limb. In a further aspect the body part is a foot, calf, thigh, leg, hand, wrist, forearm, upper arm, arm, neck, or torso.

In another aspect the pliable sheet can be configured as a wrap having a first and second end portion that are configured to be attached to each other. The attachment need not be end to end, for example one end can have an attachment area that attaches to an overlapping side of the device. The wrap can be secured around a body part. In certain aspects the body part is a residual limb, a normal limb, or a diseased limb. In a further aspect the body part is a foot, calf, thigh, leg, hand, wrist, forearm, upper arm, arm, neck, or torso. In certain aspects the wrap is secured by surrounding bandaging tape, straps, or the two ends are attached using a hook and loop attachment (e.g., VELCRO™), buckle, snap, adjustable buckle, a loop strap, tape, adhesive, or other fastener. In other aspects the pliable sheet is pressed, held and/or forced against a selected body area by means of a detachable compression cover, a pneumatic pack, compressive bandaging or wrapping, which is secure, for example by VELCRO™ stretch fabric and/or with VELCRO™ strapping or other fastener.

Certain embodiments are direct to a device where the protrusion is configured to be moveable. In certain aspects the protrusion can move towards the centerline of the device when the edge is in contact with skin. In certain aspects each protrusion is independently moveable relative to other protrusions. In other aspects two or more protrusions are positioned on a moveable surface that can be moved away from the outer edge of the pliable sheet. In certain aspects the device tapers to the distal end, i.e., the sheet is narrower at the end closest to the heart and tapers to the end most distal from the heart from a vascular perspective. In certain aspects an external force is exerted to move the protrusions toward the limb or skin. In certain aspects the movement of the protrusions is mechanically, pneumatically, or electrically controlled by the device or an external control mechanism; in such forms, the movement of the protrusions can be coordinated with the heart-beat or pulse. Coordination with the pulse of a subject can be used to minimize interference with blood flow, if needed.

The pliable sheet or lining can be manufactured using various known material(s). In certain aspects a sheet or lining described herein can be made from neoprene, nitrile, latex, rubber, foam, nylon, gel, urethane, silicone, polymer, elastomer, natural or synthetic material, or a pliable plastic material. The protrusion can be formed of a rubber, plastic, foam, nylon, gel, urethane, silicone, polymer, elastomer, natural or synthetic material, or metal.

Certain embodiments are directed to an assist device comprising a support or a support and lining, the support or the lining having an interior surface and exterior surface, the interior surface comprising a plurality of protrusions having a base proximal to the interior surface and an outer edge distal to the interior surface, the protrusions having a first acute angle relative to the inner surface positioned in the direction of desired fluid flow. As used herein, an assist device refers to a device that assists both the cardiovascular system used to transport blood and the lymphatic system that transports lymph. In certain aspects the support can be a prosthetic socket or a limb or body part casing. In certain aspects the support tapers to the distal end, i.e., the thickness of the support is narrower at the end closest to the heart and tapers to the end most distal from the heart from a vascular perspective. In a further aspect a device can further comprises an expansion gap formed between a support and its associated lining. In certain embodiments a fluid, gas, or mechanical force can be applied to the lining using electronic actuators. The force can be rhythmically applied to provide a rolling motion in the direction of desired fluid flow.

As referred to herein, an amputee is a person who has lost part of an extremity or limb such as a leg or arm, which commonly may be termed as a residual limb. Residual limbs come in various sizes and shapes with respect to the stump. That is, most new amputations are either slightly bulbous or cylindrical in shape while older amputations that may have had a lot of atrophy are generally more conical in shape. Residual limbs may further be characterized by their various individual problems or configurations including the volume and shape of a stump and possible scar, skin graft, bony prominence, uneven limb volume, neuroma, pain, edema or soft tissue configurations.

Limbs can be amputated at a number of positions. Typically, a “transtibial” amputee has been amputated between the knee and the ankle and a “transfemoral” amputee has been amputated between the knee and the hip; for the arm, the corresponding terms are transradial (below elbow) and transhumeral is elbow to shoulder. A below the knee residual limb or residuum is described as a leg having been severed below the knee terminating in a stump. The residual limb includes soft tissue as well as the femur, knee joint, and severed tibia and fibula. Along these bone structures surrounded by soft tissue are nerve bundles and vascular routes. A below the knee residual limb has its stump generally characterized as being a more bony structure while an above the knee residual limb may be characterized as including more soft tissue as well as the vascular routes and nerve bundles.

Amputees who have lost a part of their arm also have remaining vascular routes and nerve bundles as well as soft and bony tissues. The residual limb typically includes the humerus bone extending from below the shoulder to the elbow from which the radius and ulna bones may pivotally extend to the point of severance. Along the humerus bone are the biceps muscle and the triceps muscle, which still yet may be connected to the radius and the ulna, respectively.

In other aspects a subject has or has had an illness, an accident, or a bodily injury that results in immobilization of a subject or one or more limbs of a subject. For example, bed-ridden or wheelchair-bound patients; patients with immobile limbs, such as elderly, debilitated or sedated patients; and diabetics that can suffer significant damage due to circulatory deficiency.

Other embodiments of the invention are discussed throughout this application. Any embodiment discussed with respect to one aspect of the invention applies to other aspects of the invention as well and vice versa. Each embodiment described herein is understood to be an embodiment of the invention that is applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 is a cross-sectional view of one embodiment in which the inner walls of a prosthesis, orthotic, or lymphatic-treatment device are manufactured in a way that exploits the natural pressures from contacting the skin during everyday activities. The resulting pressure, promotes proximal (upward in these examples) movement of body fluids because of the shape of the protrusions. Two example shapes for the protrusions are shown (Frames 1 and 2) and for each, a view is shown when the skin is not touching the sides of the device (a) or when the skin is touching the walls of the device (b).

FIG. 2 illustrates examples of another embodiment in which the protrusion is manufactured in a way and with a material such that the protrusions can move laterally as they contact the wearer's skin. In 1-a, the protrusion (A) is hinged (B) and a spring (C) exerts lateral pressure toward the skin; in 1-b, there is no hinge, but the material used in the protrusion is flexible, moving laterally but returning to its original orientation when not in contact with the skin. The bottom two figures depict how the device shown in 1-a would perform when not touching or when touching the wearer's skin.

FIG. 3 is a cross-sectional illustration of still another embodiment in which a plate containing protrusions is mechanically moved in a direction away from the outer surface toward the wearer's skin.

FIG. 4 is a cross-sectional illustration of one embodiment of a prosthetic socket receiving a residual limb.

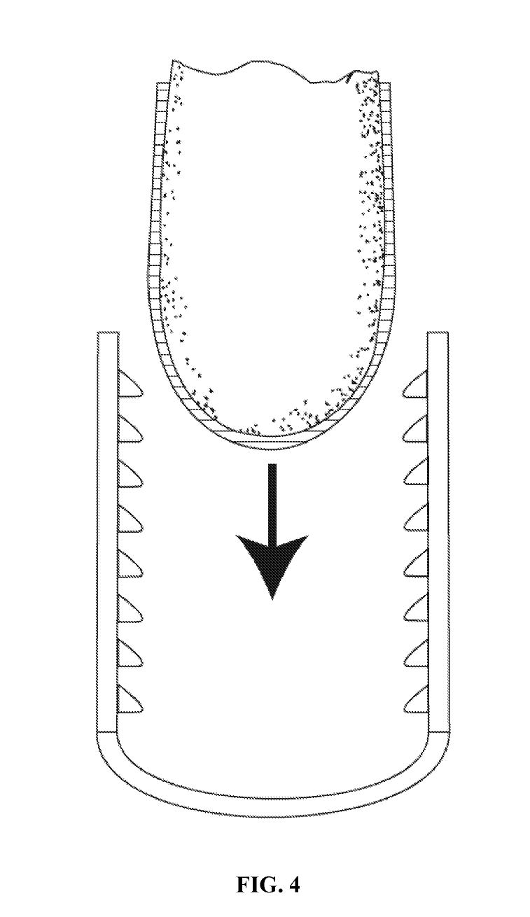

FIG. 5 is an illustration of a hydraulically actuated device to enhance fluid flow.

FIG. 6 is an illustration of a mechanically actuated device to enhance fluid flow.

### DESCRIPTION

After oxygen and nutrients are transferred to the body's cells, blood is collected and transported back to the heart through the venous system. Returning the blood to the heart from the lower extremities is more difficult because much of the pulsing pressure from the heart is reduced after the blood goes through the small capillaries and because gravity must be overcome, unless the person is lying down. Assisting this process, the veins have valves, which keep the blood moving proximally (toward the heart), i.e., to prevent it from flowing distally (away from the heart) after a pulse has pushed it forward. The importance of these valves has been known for some time; poorly functioning valves are associated with serious venous conditions/diseases. Until recently, the valves were thought to only reside in the larger veins, but recent research has shown that there are numerous such valves in the very small veins that reside more peripherally—closer to the capillaries.

There is a need for devices, drugs, or other interventions that can promote/augment/replace (i.e., assist) the process when the valves are not working correctly or when key components are missing. An example of the latter is the return of blood in a lower-limb amputee. In the normal leg, the calf muscle often is called the body's “second heart” because the mechanical pressures on veins created by contraction/relaxation of the calf muscles (e.g., while walking), can provide the force necessary to push the blood proximally (i.e., force from the calf muscle exerted perpendicular to a healthy vein is translated into proximal movement of the blood, partly because the venous valves prevent distal movement). In a lower extremity amputee (e.g., a transtibial amputee), such muscle activity is dramatically reduced—if not eliminated—resulting in potentially poorer blood return, even if the valves are working properly. Hence, any assistance in moving the blood proximally back towards the heart would be beneficial to an amputee or anyone whose venous valves are not functioning properly. The present method and apparatus replace or augment the perpendicular pressures, which normally are applied to veins and lymph vessels by muscle activity, with similar pressures applied by an external apparatus as described herein. In addition, research has suggested that it is intermittent pressure, as opposed to constant pressure, that is the most effective method for moving fluids in body tissues, so various aspects of the invention attempt to maximize the opportunity for a variety of intermittent external pressures to be applied to the tissue.

A parallel situation exists in the lymphatic system. Valves in the lymph vessels keep the lymph moving proximally, and this process is essential in removing waste materials from interstitial fluid. The perpendicular pressure source for the lymphatic vessels comes from the contraction/relaxation of neighboring muscles (as in the venous system), and from periodic contractions in the lymph vessels themselves. Failure of the lymphatic system to properly move lymph proximally can result in lymphedema, a potentially serious condition. Hence, any assistance in moving the lymph proximally would be beneficial to a patient with edema or lymphedema.

Various embodiments address the general objective of moving blood or lymph proximally. In certain aspects the embodiments are a “passive” form. In certain aspects the device can be positioned on the inside wall of a prosthetic device, orthotic device, or limb encasement (for lymphedema) in a way that promotes proximal fluid movement. Certain embodiments are designed to be worn while a patient moves naturally during activities of daily living, and take advantage of the natural forces that occur during such activities (e.g., while walking). Other embodiments take a more dynamic approach to applying such forces to a device worn by the patient during daily activities.

Embodiments are designed to increase proximal flow of the blood or lymph by applying pressure perpendicular to the veins or lymphatic vessels in a body extremity. Additional pressure augments the natural forces applied to the vessels (e.g., pressure on both veins and lymphatic vessels from muscle action and pressure created inside the lymphatic vessels by the periodic contraction of the smooth muscles in their walls). While additional pressure will not repair broken/missing valves, it can improve the efficiency of partially functioning valves by providing more fluid throughput and by maximizing the effectiveness of any normally functioning valves (e.g., by driving the fluid far enough proximally that it passes a normally functioning valve).

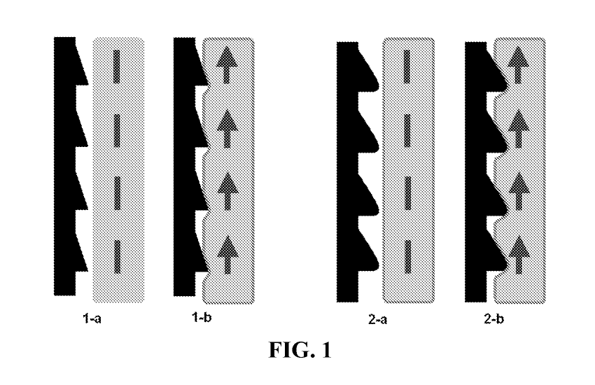

An example of one embodiment is illustrated in FIG. 1. Both frames show a cross-sectional view of the wall of a prosthesis/orthotic/lymphedemic device having protrusions on the interior surface of the device and the skin of the wearer, when the device is not touching the wearer's skin (1-a and 2-a) and when natural body movement causes the skin to touch the device (1-b and 2-b). The two forms differ in the shape of the manufactured “protrusions,” but the point is that they both provide pressure perpendicular to proximal flow (upward) when the skin touches them and their shape systematically pushes the fluid proximally as more pressure is applied because of their wedge-like shape. Other methods have been taught where perpendicular pressure is indiscriminately applied to the limb, which may apply pressure on the fluid to move it distally, potentially damaging healthy valves located distal to the point of pressure. Research has suggested that it is intermittent pressure, as opposed to constant pressure, that is the most effective method for moving fluids in body tissues.

In certain embodiments of the devices described herein the protrusions can be manufactured in concentric circles, in spirals, or individually at various locations. In certain aspects the presence, shape, location, and prominence of the protrusions are based on what is known of the physiology of the vascular and lymphatic system in the affected regions (e.g., there are fewer and less prominent protrusions in the anterior side of the tibia (so as to not provide such pressure on that bone), and more, and more prominent protrusions at anatomical sites where major veins or lymph vessels are located. The shape of the protrusions can help keep the device in position on a subject. As shown in FIG. 1, the hook- or wedge-like protrusions reduce the chance of the device slipping downward. Applicants note that while some of the figures and discussion make reference to the edges of the apparatus touching the “skin,” it should be noted that this should be interpreted as directly touching the skin or indirectly touching the skin through a prosthetic liner, covering, sleeve, sock, etc. Furthermore, while the protrusions shown in the figures are all of the same height, in some embodiments the actual heights of the protrusions might change from site to site as a function of the peripheral depth of the target blood or lymph vessel(s) those sites.

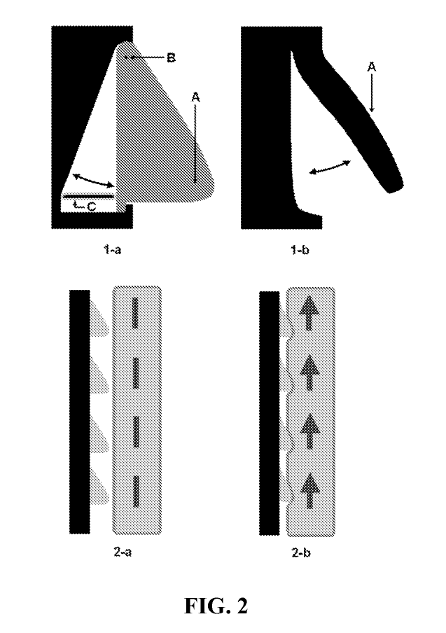

In certain embodiments the protrusions can be configured to allow some lateral movement (with the most movement allowed in the distal “thickest” portion of the protrusion). FIG. 2 presents two representative examples of this approach (1-a and 1-b). In 1-a, the protrusion (A) is hinged at a pivot point (B) to the side of the device, and a spring or other component with elastic properties (C) is provided to force the protrusion against the skin as the wearer performs everyday activities. In form 1-b, there is no specific pivot point, but the protrusion is manufactured in a way (e.g., as a flap) and with a material that allows inherent lateral “spring-like” movement of the protrusion as it comes in contact with the skin. The pivoting approach depicted in 1-a is further shown in the context of its relationship with the prosthetic device and the wearer's skin when the protrusion is not touching the wearer's skin (2-a) and when it is touching the skin (2-b). In this aspect the protrusions are flexible and will cause less irritation on the skin at the points where contact is made. Such embodiment can be used when the subject suffers from dermatological or neurological problems.

In certain aspects a device described herein can be used in a prosthesis worn by a lower-limb amputee to promote blood and lymph flow. In other aspects a device as described herein can be used in other populations/settings such as lower-limb non-amputees, upper limb amputees, upper limb non-amputees, patients with lymphedema, etc.

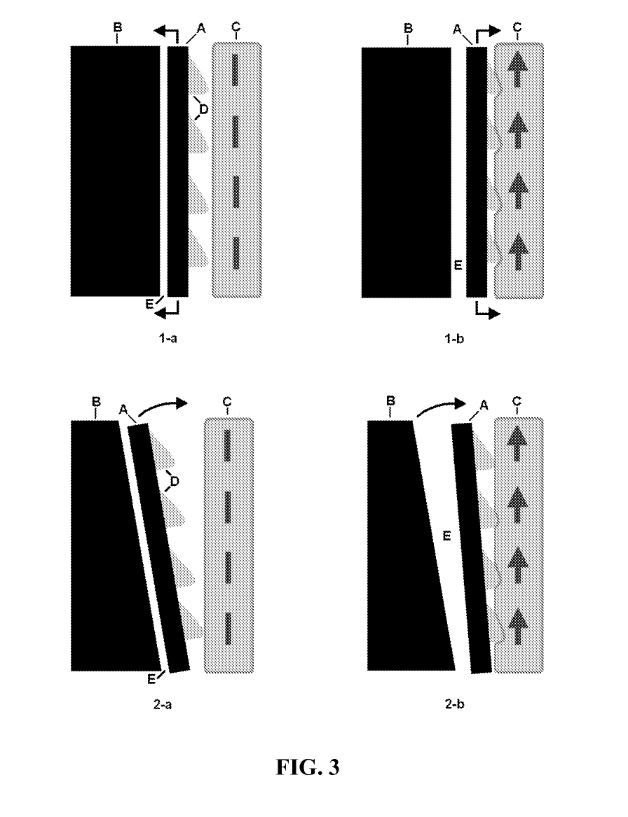

In still other embodiments a more dynamic apparatus is designed to augment the natural forces exerted while patients move about and perform activities of daily living. In certain aspects a device incorporates a plate with a surface that contains protrusions as described above and a mechanism for periodically forcing that surface against a subject's skin. In certain aspects force can be systematically applied in a controlled manner. FIG. 3 provides examples of the more dynamic form of the device. In both examples (top and bottom), cross-sectional illustrations depict how a plate (A) with one surface containing protrusions (D—as described above) is mechanically moved in a direction away from the exterior surface of the device or a supporting device such as a prosthetic socket or encasing (B) and toward the wearer's skin (C). In the top example, the plate is moved from an initial location close to the socket (as shown in 1-a) toward the user's skin in a direction perpendicular to the surface of the user's skin (as shown in 1-b). In the bottom example, the plate is hinged to the socket or casing at distal end, so that its movement from an initial location close to the socket (as in 2-a), to a later location contacting the surface of the user's skin (as in 2-b), initially applies more pressure distally and with pressure being progressively applied in the proximal direction. The distal pivot point in the design depicted in FIGS. 3 (2a) and (2b) is intended to further increase the “milking” movement of fluids because the individual movement effected by each protrusion is joined by a general distal-to-proximal movement for the entire surface. In both cases, movements of the plate toward the skin as illustrated from 1-a to 1-b and from 2-a to 2-b are followed by movements back to the starting position from 1-b to 1-a and from 2-b to 2-a, and the resulting cycles are repeated over time.

Movements of the plates can be effected by mechanical devices (e.g., actuators, solenoids, etc.) or by introducing/removing pressure in the volume between the socket and the plate (E—e.g., air or fluid pressure/vacuum created by an external source or by capturing energy from the user's action, as commonly utilized in current “vacuum-assisted” prostheses to help keep the prosthetic device on). Cyclic movement toward and away from the user's skin can be periodic, can be independent of the actions of the user, can be synchronized with user's body movements, or can be synchronized with the wearer's pulse.

In the case of a prosthetic device for a lower limb, it could be advantageous to initiate a sequence of applying pressure to the skin (i.e., 1-a to 1-b and 2-a to 2-b) during the very late stage of stance and very early stage of swing, so that the resulting pressure between the socket and the skin is maximal during the subsequent swing phase, helping hold the socket on while gravity and centrifugal forces work to doff the socket. Movement of the plates away from the skin (i.e., 1-b back to 1-a and 2-b back to 2-a) can be initiated by heel strike of the residual limb and the plate remains in a neutral location during most of the stance phase (when the patient's weight is sufficient to keep the socket in place).

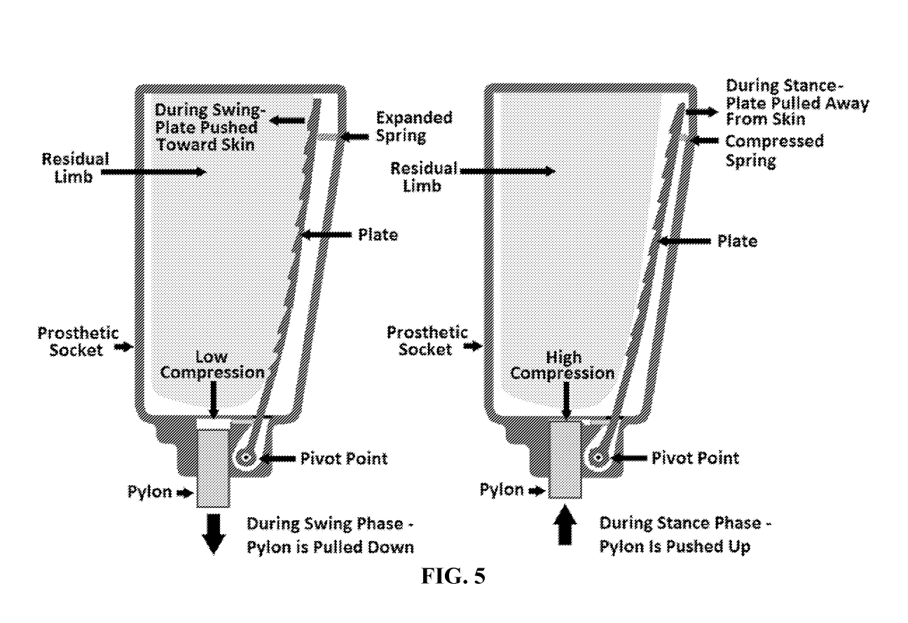

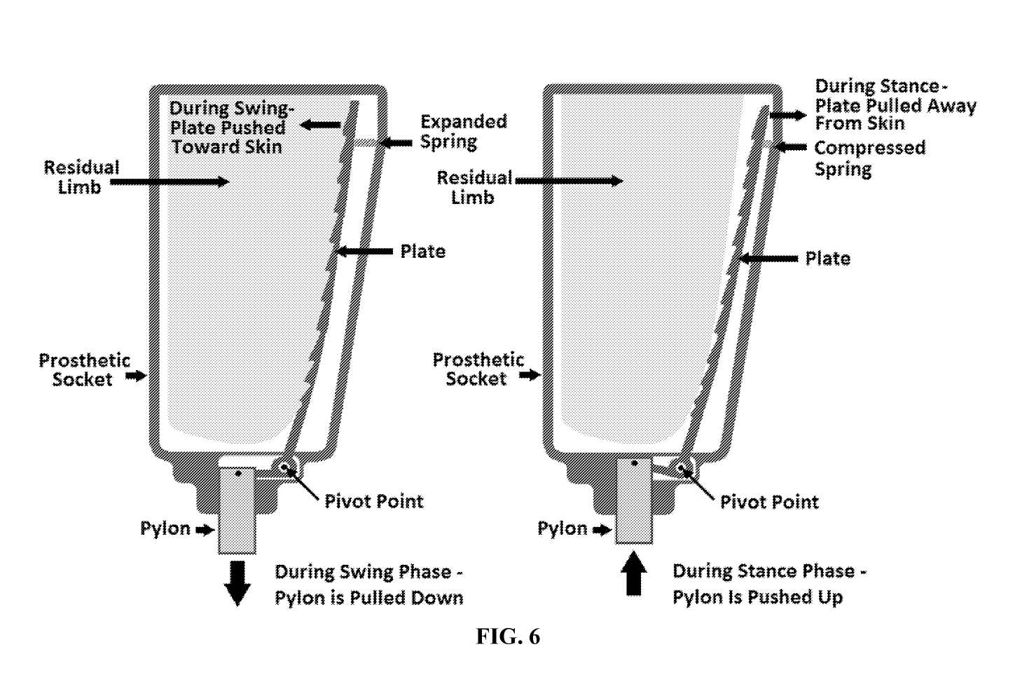

FIG. 5 and FIG. 6 illustrate the operation of hydraulically or mechanically actuated flow enhancement prosthesis as a wearer moves. During leg swing the pylon is under low compression and is positioned in an extended configuration, moving outward from the base of the prosthesis. Movement of the pylon reduces the hydraulic or mechanical force applied to the plate that is operatively coupled to the base at a pivot point that allows movement of the plate to or from the surface of the residual limb that is positioned in the prosthetic socket of the prosthesis. The reduction in force allows the plate move toward the long axis of the prosthesis and contact the residual limb. During standing the pylon is under high compression and the force causes the pylon to contract or move inward. The inward movement exerts hydraulic or mechanical force on the plate causing the plate to move away from the residual limb and reduce the pressure of contact between the residual limb and the plate.

Alternatively, activation of the mechanism that forces the plate toward the skin could be synchronized with the wearer's pulse so that, for example, the resulting proximal movement of blood back toward the heart is consistent with the natural cardiovascular cycle and so that pressures are not being externally applied which could interfere with the natural arterial proximal-to-distal movement of blood toward the peripheral regions of the limb.

1. A fluid flow assist device having:
a top edge, a bottom edge positioned opposite the top edge, two side edges connecting the top and bottom edges, and a surface having an exterior and interior surface;
the interior surface comprising a plurality of protrusions having a base proximal to the interior surface and an apex distal to the interior surface, each protrusion having a top and bottom surface with the top surface of the protrusions facing the top edge of the device and the bottom surface of the protrusion facing the bottom edge of the device, the bottom surface of the protrusions is substantially perpendicular to the interior surface and the top surface of the protrusions slopes towards the top edge from the apex to the interior surface forming a wedge shape, wherein as the interior surface of the device moves toward and contacts a body part positioned in the lumen of the device the apex of the protrusion makes first contact with the body part followed progressively by the slope of the top surface of the protrusions where a progressively increasing pressure is exerted by the protrusion on the body fluid contained in the body part moving the body fluid towards the top edge of the device.

2. The device of claim 1, wherein the device is a prosthetic socket or a prosthetic socket liner.

3. (canceled)

4. The device of claim 1, wherein the device is a configured as a cover that is pulled over or positioned around a body part.

5. The device of claim 4, wherein the body part is a residual limb.

6. The device of claim 4, wherein the body part is a foot, calf, thigh, leg, hand, wrist, forearm, upper arm, or arm.

7. (canceled)

8. The device of claim 4, wherein the covering is configured as a wrap having a first and second side edges that are configured to be attached to each other to form the lumen.

9. The device of claim 8, wherein the first and second side edges comprise a hook and loop portion for attaching the side edges.

10. The device of claim 1, wherein the protrusions are configured to be moveable towards the surface of the device when the edge is in contact with skin or a limb covering.

11. The device of claim 10, wherein the movement of the protrusions is coordinated with the heart-beat.

12. The device of claim 1, wherein each protrusion is independently moveable relative to other protrusions.

13. The device of claim 1, wherein the protrusions are configured as linear bands, spiral bands, or an array of protrusions.

14. The device of claim 1, wherein the size of the protrusions are heterogeneous.

15. The device of claim 1, wherein spatial distribution of the protrusions is irregular.

16. The device of claim 1, wherein the device is an orthotic or an orthotic liner.

17. (canceled)

18. The device of claim 16, wherein the orthotic is a configured as a covering that is pulled over or positioned around a body part.

19. The device of claim 18, wherein the body part is a foot, calf, thigh, leg, hand, wrist, forearm, upper arm, or arm.

20. (canceled)

21. The device of claim 16, wherein the orthotic is configured as a wrap having a first and second side edges that are configured to be attached to each other to form the lumen.

22. The device of claim 16, wherein the protrusions are configured as linear bands, spiral bands, or an array of protrusions.

23. (canceled)

24. The device of claim 16, wherein spatial distribution of the protrusions is irregular.

25. A fluid assist device comprising:
a body that forms a lumen to receive a body part having a top and a bottom; and
a fluid assist surface having an interior surface facing the lumen of the body, the interior surface having a plurality of protrusions, each protrusion having a top and bottom surface, the top surface of the protrusion facing the top of the body and the bottom surface of the protrusion facing the bottom of the body, wherein the top surface slopes towards the top of the body and the bottom surface is substantially perpendicular to the interior surface forming a wedge shape protrusion that is configured to provide a progressively increasing pressure exerted on body fluids to move the body fluid towards the top of the device.

26.-30. (canceled)
